SEQUENCE LISTING (15 sequences)

SEQ ID NO: 1 - DNA, 93 bp, synthetic construct
  misc_feature 1..93: note = LacZ (deltaM15) Deletion Fragment
  source 1..93: mol_type = other DNA
gttttacaacgtcgtgactgggaaaaccctggcgttacccaacttaatcgccttgcagcacatccccctttcgccagctggcgtaatagcgaa

SEQ ID NO: 2 - DNA, 54 bp, synthetic construct
  misc_feature 1..54: note = Primer E461A N20 F
  source 1..54: mol_type = other DNA
tggtcgctggggaatgaatcgttttagagctagaaatagcaagttaaaataagg

SEQ ID NO: 3 - DNA, 49 bp, synthetic construct
  misc_feature 1..49: note = Primer E461A N20 R
  source 1..49: mol_type = other DNA
gattcattccccagcgaccaactagtattatacctaggactgagctagc

SEQ ID NO: 4 - DNA, 54 bp, synthetic construct
  misc_feature 1..54: note = Primer E537A N20 F
  source 1..54: mol_type = other DNA
tcgcgtgggcgtattcgcaagttttagagctagaaatagcaagttaaaataagg

SEQ ID NO: 5 - DNA, 48 bp, synthetic construct
  misc_feature 1..48: note = Primer E537A N20 R
  source 1..48: mol_type = other DNA
ttgcgaatacgcccacgcgaactagtattatacctaggactgagctag

SEQ ID NO: 6 - DNA, 54 bp, synthetic construct
  misc_feature 1..54: note = Primer deltaM15 N20 F
  source 1..54: mol_type = other DNA
cgtcgtgactgggaaaacccgttttagagctagaaatagcaagttaaaataagg

SEQ ID NO: 7 - DNA, 53 bp, synthetic construct
  misc_feature 1..53: note = Primer deltaM15 N20 R
  source 1..53: mol_type = other DNA
gggttttcccagtcacgacgactagtattatacctaggactgagctagctgtc

SEQ ID NO: 8 - DNA, 43 bp, synthetic construct
  misc_feature 1..43: note = Primer E461/537 F
  source 1..43: mol_type = other DNA
tggtcggcttacggcggtgattaagcttagatctattaccctg

SEQ ID NO: 9 - DNA, 47 bp, synthetic construct
  misc_feature 1..47: note = Primer E461/537 R
  source 1..47: mol_type = other DNA
tctgcttcaatcagcgtgccgtcggaattcaaaaaaagcaccgactc

SEQ ID NO: 10 - DNA, 43 bp, synthetic construct
  misc_feature 1..43: note = Primer pT-N20-F
  source 1..43: mol_type = other DNA
cagggtaatagatctaagcttaatcaccgccgtaagccgacca

SEQ ID NO: 11 - DNA, 47 bp, synthetic construct
  misc_feature 1..47: note = Primer pT-N20-R
  source 1..47: mol_type = other DNA
gagtcggtgctttttttgaattccgacggcacgctgattgaagcaga

SEQ ID NO: 12 - DNA, 48 bp, synthetic construct
  misc_feature 1..48: note = Primer lacZ (deltaM15) F
  source 1..48: mol_type = other DNA
accgagtcggtgctttttttgaattcaatgcgcgccattaccgagtcc

SEQ ID NO: 13 - DNA, 52 bp, synthetic construct
  misc_feature 1..52: note = Primer lacZ (deltaM15) R
  source 1..52: mol_type = other DNA
gataacagggtaatagatctaagcttccgacccagcgcccgttgcaccacag

SEQ ID NO: 14 - DNA, 20 bp, synthetic construct
  misc_feature 1..20: note = wacJ N20
  source 1..20: mol_type = other DNA
gtggtgttccagatgttggg

SEQ ID NO: 15 - DNA, 20 bp, synthetic construct
  misc_feature 1..20: note = fucK/I N20
  source 1..20: mol_type = other DNA
ttgagttggtgcgtttgttg

(19) United States
(12) Patent Application Publication
(10) Pub. No.: US 2024/0309415 A1
(43) Pub. Date: Sep. 19, 2024

(54) A GENETICALLY ENGINEERED BACTERIUM WITH LACZ INACTIVATION AND ITS USE IN PRODUCING HUMAN MILK OLIGOSACCHARIDES

(71) Applicant: SYNAURA BIOTECHNOLOGY (SHANGHAI) CO., LTD., Shanghai (CN)

(72) Inventors: Qi JIAO, Shanghai (CN); Zhenhua TIAN, Shanghai (CN); Shu WANG, Shanghai (CN); Zhanbing CHENG, Shanghai (CN); Xiaolan XU, Shanghai (CN); Fei YAO, Shanghai (CN); Miao LI, Shanghai (CN); Hong XU, Shanghai (CN); Chenxi HUANG, Shanghai (CN); Yurou LIU, Shanghai (CN)

(73) Assignee: SYNAURA BIOTECHNOLOGY (SHANGHAI) CO., LTD., Shanghai (CN)

(21) Appl. No.: 18/576,585

(22) PCT Filed: Oct. 11, 2022
(86) PCT No.: PCT/CN2022/124632

§ 371 Date: Jan. 4, 2024

(30) Foreign Application Priority Data
Nov. 26, 2021 (CN) 202111422436.6

(51) Int. Cl. C12P 19/04; C12N 1/20; C12R 1/19

(52) U.S. Cl. CPC C12P 19/04; C12N 1/205; C12N 2500/12; C12N 2500/34; C12N 2500/35; C12N 2500/74; C12R 2001/19

(57) ABSTRACT

The present invention discloses a genetically engineered bacteria, which is E. coli integrated with lysogenic λDE3, and lacZ gene is completely inactivated, but does not affect exogenous protein expression of the genetically engineered bacteria. The present invention also discloses a method for culturing the genetically engineered bacteria, and a method for preparing human milk oligosaccharides using the same, and use of the genetically engineered bacteria. The genetically engineered bacteria of the present invention can efficiently produce human milk oligosaccharides, such as 2′-fucosyllactose, and have wide industrial application prospects.

### FIELD OF THE INVENTION

The present invention belongs to the field of genetic engineering, and relates to a genetically engineered bacteria in which the lacZ gene encoding β-galactosidase is inactivated. and its use in producing human milk oligosaccharide.

### BACKGROUND OF THE INVENTION

Human milk is composed of a mixture of carbohydrates, proteins, lipids, hormones and trace elements, which not only provides the nutrients required for the growth and development of infants, but also provides protective agents such as immunoglobulins. In addition to this, human milk also contains a series of complex oligosaccharides with protective properties-human milk oligosaccharides.

Human milk oligosaccharides (HMOs) are a type of non-digestible carbohydrates in human milk with complex structure, the content of which is 22-24 g/L in human colostrum and 5˜12 g/L in normal human milk, it is the third largest solid content in human milk after fat and lactose. By stimulating the growth of intestinal probiotics such as bifidobacteria and lactobacilli in neonates and balancing the development of gut microbiota, HMOs may play an important role in regulating the postnatal immune system of neonates, and as functional components of advanced infant formulas, HMOs are very important. In addition, HMOs can inhibit the adhesion of pathogens to glycans on the surface of epithelial cells, thereby limiting the virulence of some pathogens.

There are more than 200 different oligosaccharides in human milk, and the structures of 115 kinds of human milk oligosaccharides have been identified. Based on the monosaccharide structural units that make up HMOs, HMOs can be classified into three types: neutral fucosyllactose, acid sialyllactose, and neutral afucosylated lactose. Whole cell biosynthesis is a preparation method of HMOs, in which lactose needs to be added as a raw material, and β-galactosidase in cells will decompose lactose, thereby reducing the utilization of lactose and affecting the yield of HMOs, therefore, the production of HMOs mostly adopts the method of knocking out or partially knocking out lacZ (β-galactosidase gene) in host cells, to reduce the activity of intracellular β-galactosidase, thus increasing the yield of HMOs. Currently, lacZ (ΔM15)-deficient cloned hosts are mainly used in production, such as top10, DH5α, JM109, etc. However, these host types cannot completely eliminate the expression of lacZ, and because of the lack of T7 RNA polymerase gene, it is difficult for them to express vectors containing T7 strong promoter series, thus limiting the production of HMOs.

In many reports on the production of 2′-fucosyllactose (2′-FL), such as CN109790559A, Journal of Biotechnology 210 (2015) 107-115, etc., lacZ (ΔM15)-deficient hosts (without λDE3) are adopted as chassis cells, but β-galactosidase still retains 3% activity in such cells, which may be a hidden danger of lactose degradation in fermentation production (Journal of Biotechnology 210 (2015) 107-115). The reason is E. coli has very low levels of intracellular β-galactosidase in the absence of lactose, about no more than 5 molecules per cell, and the number of β-galactosidase molecules in per cell can increase to 5000 in only 2-3 minutes after the addition of lactose. The increased number of enzyme molecules leads to an increase in the overall enzymatic activity, resulting in an increased amount of lactose degradation, thereby reducing production efficiency.

Since the lacZ (ΔM15)-deficient cloned host lacks T7 RNA polymerase gene, some new host types were later derived, such as DH5α (λDE3), JM109 (λDE3), etc. Due to the integration of lysogenic λDE3, these hosts obtains T7 RNA polymerase genes and can be used in T7 strong promoter expression systems. However, the study by Angela Zhang et al. (Metabolic Engineering 66 (2021) 12-2) shows that in BL21 Star (λDE3), when the lacZ gene is completely deleted, it will lead to the loss of exogenous protein expression function. After sequencing, it was found that in attB integration site, the λDE3 lysogen containing PlacUV5: lacZα-T7rnap was excised. The present inventors also found this phenomenon by knocking out or partially knocking out the lacZ gene on the basis of JM109 (λDE3). This indicates that in the host containing lysogenic λDE3 with the knockout or partial knockout of the lacZ gene, loss of exogenous protein expression function is a common phenomenon.

Therefore, when using colorectal cells integrated with lysogenic λDE3 as chassis, the complete inactivation of the lacZ gene and the stable expression of exogenous proteins during the production process of HMOs using lactose as acceptor is an economic and scientific issue to study.

### DETAILED DESCRIPTION OF THE INVENTION

In order to solve the technical problem in the prior art, i.e., there is no productive strains that effectively produce HMOs without the loss of exogenous protein expression function due to the lacZ gene knockout, the present invention provides a β-galactosidase inactivated strain and its use in the production of human milk oligosaccharides.

In order to solve the above-mentioned technical problems, one of the technical solutions of the present invention is to provide a genetically engineered bacteria, which is E. coli integrated with lysogenic λDE3, and the lacZ gene is completely inactivated or substantially inactivated, but the exogenous proteins expression in genetically engineered bacteria is not affected. It is well known to those skilled in the art that the complete inactivation or substantial inactivation refers to complete or almost no detectable activity in conventional enzymatic activity detection experiments; or even if data higher than the background signal is detected, the activity is negligible compared to wild-type enzyme.

In some preferred embodiments. β-galactosidase encoded by said lacZ gene exhibits E461A and/or E537A mutations compared to β-galactosidase encoded by wild-type lacZ. Preferably, codon for amino acid A is GCG. In the present invention, the difference can be due to mutating the wild-type β-galactosidase to form E461A and/or E537A; it can also be due to using other β-galactosidase as a template, on which one or more mutations occur, and finally form a mutant similar to E461A and/or E537A compared with the wild-type β-galactosidasc.

In some more preferred embodiments, the wacJ, fucK and fucI genes are further knocked out in the genetically engineered bacteria.

In some preferred embodiments, the genetically engineered bacteria further comprise fucT gene and fkp gene.

In some more preferred embodiments, the fucT gene is fucT gene derived from Helicobacter pylori, and the fkp gene is fkp gene derived from Bacteroides fragilis.

In some preferred embodiments, the fucT gene is the gene with GenBank accession number AF076779, and the fkp gene is the gene with GenBank accession number AAX45030.1.

In some more preferred embodiments, the fucT gene and the fkp gene are respectively inserted into different backbone plasmids pETduet-1, or inserted into same backbone plasmid pETduct-1 at the same time, and are present in the genetically engineered bacteria as a recombinant expression vector.

In some preferred embodiments, the starting strain is E. coli DH5α (λDE3), E. coli BL21 (λDE3), E. coli BL21 Star (DE3), or E. coli JM109 (λDE3).

In order to solve the above technical problems, the second technical solution of the present invention is to provide a method for culturing genetically engineered bacteria, comprising culturing the genetically engineered bacteria according to one of the technical solutions of the present invention in a culture medium.

In some preferred embodiments, the medium is E. coli conventional medium, preferably LB, SOB, SOC, 2×YT, TB, SB medium.

In order to solve the above technical problems, the third technical solution of the present invention is to provide a method for preparing human milk oligosaccharide, which uses the genetically engineered bacteria according to one of the technical solutions of the present invention to ferment the substrate.

In some preferred embodiments, the human milk oligosaccharide is selected from 2′-fucosyllactose, 3-fucosyllactose, 3′-sialyllactose, 6′-sialyllactose, lactose-N-tetrasaccharide, lactose-N-neotetraose, lactose-N-hexasaccharide, lactose-N-fucopentose I, lactose-N-fucopentose II, lactose-N-fucopentaose III, and lactose-N-fucopentaose V.

In some more preferred embodiments, the human milk oligosaccharide is 2′-fucosyllactose, and the substrates include L-fucose and lactose. In order to solve the above technical problems, the fourth technical solution of the present invention is to provide a method for preparing 2′-fucosyllactose, using the genetically engineered bacteria according to one of the technical solutions of the present invention, adding L-fucose and lactose into the fermentation medium for fermentation to obtain the 2′-fucosyllactose;

In some preferred embodiments, the fermentation medium for culturing the genetically engineered bacteria includes 20 g/L glycerol or glucose, 10 g/L peptone, 5 g/L yeast powder, and 10 g/L NaCl.

Preferably, when the genetically engineered bacteria are cultured to OD₆₀₀=0.5-1.0, preferably 0.6-0.8, a final concentration of 0.1-0.3 mM IPTG such as 0.1 mM IPTG, 5 g/L L-fucose, and 10 g/L lactose are added.

In order to solve the above technical problems, the fifth technical solution of the present invention is to provide use of the genetically engineered bacteria according to one of the technical solutions of the present invention in the production of human milk oligosaccharide inoculants.

In some preferred embodiments, the human milk oligosaccharide is selected from 2′-fucosyllactose, 3-fucosyllactose, 3′-sialyllactose, 6′-sialyllactose, lactose-N-tetrasaccharide, lactose-N-neotetraose, lactose-N-hexasaccharide, lactose-N-fucopentose I, lactose-N-fucopentose II, lactose-N-fucopentaose III, and lactose-N-fucopentaose V.

In some more preferred embodiments, the human milk oligosaccharide is 2′-fucosyllactose.

In order to solve the above technical problems, the sixth technical solution of the present invention is to provide use of the genetically engineered bacteria according to one of the technical solutions of the present invention in producing 2′-fucosyllactose.

On the basis of conforming to common knowledge in the art, the above preferred conditions can be combined arbitrarily to obtain preferred embodiments of the present invention.

The reagents and raw materials used in the present invention are all commercially available.

The positive progressive effect of the present invention is:

To provide a genetically engineered bacterium in which the lacZ gene is completely inactivated, for example, after E461A and/or E537A mutation occurs, β-galactosidase activity is lost, but the exogenous protein expression function is maintained, and human milk oligosaccharide, for example 2′-fucosyllactose, can be efficiently produced.

### DESCRIPTION OF THE DRAWING

FIG. 1 is a schematic diagram of the pathway of producing 2′-FL according to the present invention.

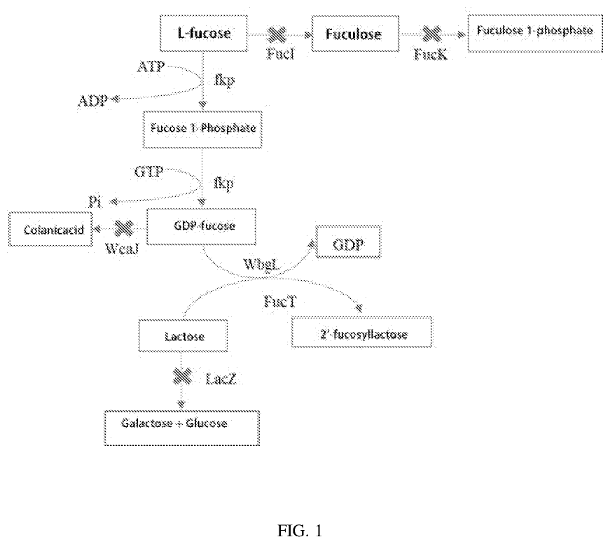

### EXAMPLES

The present invention is further described below by way of examples, but the present invention is not limited to the scope of the described examples. The experimental methods with no specific conditions in the following examples are selected according to conventional methods and conditions, or according to product insert.

In the examples, a high performance liquid chromatography (HPLC) system (SHIMADZU LC-20AD XR) was used to quantitatively detect the synthesis of 2′-FL in the fermentation broth of recombinant E. coli, and the concentration of 2′-FL and the substrate lactose in the fermentation broth was determined by HP-Amide column (Sepax, 4.6×250 mm 5 μm). The HPLC detector was a differential detector, the detection temperature of the chromatographic column was set to 35° C., the mobile phase was eluted by acetonitrile: water=68: 32, and the detection flow rate was 1.4 mL/min.

The BL21 (λDE3) strain was purchased from Novagen Company, Cat. #69450-M; pTargetF plasmid, pETduct-1 plasmid, and pCas9-containing plasmid are all commercially available.

### Example 1 Functional Inactivation of lacZ Gene

(1) Preparation of E. coli BL21 (λDE3) Competent Cells Containing pCas9 Plasmid

pCas9 plasmid was transfected into E. coli BL21 (λDE3), which was coated on 50 μg/mL kanamycin LB plates, cultured at 30° C. for 12 h, and single colonies were picked and inoculated in fresh LB liquid medium (containing kanamycin with final concentration 50 g/mL), overnight cultured at 30° C., 220 r/min; the overnight cultured bacterial solution was transferred to 50 ml LB medium at an inoculum of 1%. and when the bacterial OD₆₀₀ reached 0.2, arabinose was added to a final concentration of 2 g/L to induce pCas9 plasmid to express recombinase; the induction was continued at 30° C. for 2 h, the cells were harvested and bathed in ice-water for 10 min; the cells were washed twice with pre-chilled sterile water, and the supernatant was discarded, the bacterial cells were resuspended with 500 μL of pre-chilled 10% glycerol, dispensed 50 μL each, immediately frozen in liquid nitrogen, and stored in a refrigerator at minus 80° C. In this way, E. coli BL21 (λDE3) competent cells with pCas9 plasmid were prepared.

### (2) Preparation of Plasmid Vector for Gene Mutation

the lacZ gene sequence (GenBank: U00096.3) was obtained, and N20 sequence that specifically targets the lacZ gene was designed. Using different primer pairs “(X)N20F and (X)N20R” (X=E461A, E537A, ΔM15), N20 was ligated into the vector pTargetF to obtain three different pT-N20 plasmids. On this basis, the homology arms corresponding to different mutations were cloned using primer pairs “E461/537 Primer F and E461/537 Primer R” and “lacZ(ΔM15)F and lacZ(ΔM15)R” respectively, and the linearized vector was cloned using primer pair “pT-N20-F and pT-N20-R”, and finally, using seamless cloning technology it was ligated into the corresponding pT-N20 plasmid vector to obtain pT-E461A. pT-E537A. and pT-ΔM15 vectors. The above primer sequences are shown in Table 1.

The designed codon for E461A mutation is GAA461GCG; the designed codon for E537A mutation is GAA537GCG.

The lacZ (ΔM15) deletion fragment (SEQ ID NO: 1) is:
“gttttacaacgtcgtgactgggaaaaccctggegttacccaacttaategccttgcagcacatccccctttcgccagctggc gtaatagcgaa”.

The three plasmids obtained above were electroporated into the competent E. coli BL21 (λDE3) containing pCas9 plasmid, and with 800 μL of LB medium added, cultured at 30° C. and 220 r/min for 2 h. coated on plates containing kanamycin (50 μg/mL) and spectinomycin (50 μg/mL), incubated overnight at 30° C. The success of gene mutation and deletion was confirmed by picking a single colony for PCR verification and DNA sequencing verification. Finally, three BL21(λDE3) host cells (ie chassis cells) containing different genotypes were obtained, as shown in Table 2, i.e., BL21(λDE3)lacZ(ΔM15), BL21(λDE3)lacZ(E461A), BL21(λDE3)lacZ (E537A), respectively.

TABLE 1
lacZ inactivation related primer sequences

| | | SEQ ID |
|---|---|---|
| Primer Name | Primer Sequence | NO: |
| E461A N20 F | tggtcgctggggaatgaatc | 2 |
| | gttttagagctagaaatagc | |
| | aagttaaaataagg | |
| E461A N20 R | gattcattccccagcgacca | 3 |
| | actagtattatacctaggac | |
| | tgagctagc | |
| E537A N20 F | tcgcgtgggcgtattcgcaa | 4 |
| | gttttagagctagaaatagc | |
| | aagttaaaataagg | |
| E537A N20 R | ttgcgaatacgcccacgcga | 5 |
| | actagtattatacctaggac | |
| | tgagctag | |
| ΔM15 N20 F | cgtcgtgactgggaaaaccc | 6 |
| | gttttagagctagaaatagc | |
| | aagttaaaataagg | |
| ΔM15 N20 R | gggttttcccagtcacgacg | 7 |
| | actagtattatacctaggac | |
| | tgagctagctgtc | |
| E461/537 primer F | tggtcggcttacggcggtga | 8 |
| | ttaagcttagatctattacc | |
| | ctg | |
| E461/537 primer R | tctgcttcaatcagcgtgcc | 9 |
| | gtcggaattcaaaaaaagca | |
| | ccgactc | |
| pT-N20-F | cagggtaatagatctaagct | 10 |
| | taatcaccgccgtaagccga | |
| | cca | |
| pT-N20-R | gagtcggtgctttttttgaa | 11 |
| | ttccgacggcacgctgattg | |
| | aagcaga | |
| lacZ (ΔM15) F | accgagtcggtgcttttttt | 12 |
| | gaattcaatgcgcgccatta | |
| | ccgagtcc | |
| lacZ (ΔM15) R | gataacagggtaatagatct | 13 |
| | aagcttccgacccagcgccc | |
| | gttgcaccacag | |

TABLE 2
Chassis cells

| Chassis Cell ID | Genetype |
|---|---|
| Chassis 1 | BL21 (λDE3) lacZ (ΔM15) |
| Chassis 2 | BL21 (λDE3) lacZ (E461A) |
| Chassis 3 | BL21 (λDE3) lacZ (E537A) |

### Example 2 Construction of HMO Chassis Cells and Related Vectors

### (1) Construction of Chassis Cells

On the basis of the three chassis cells of Example 1, wacJ, fucK and fucI genes were knocked out. The different N20 sequences are shown in Table 3 below. Experimental methods and technique are referred to Example 1.

TABLE 3
N20 sequences with wcaJ and
fuck/I knockout

| | N20 Sequence | SEQ ID NO: |
|---|---|---|
| wacJ N20 | gtggtgttccagatgttggg | 14 |
| fucK/I N20 | ttgagttggtgcgtttgttg | 15 |

Finally, three new different chassis cells were produced, as shown in Table 4 below.

TABLE 4
Chassis cells

| Chassis Cell ID | Genetype |
|---|---|
| Chassis 4 | BL21 (λDE3) lacZ (ΔM15) ΔwacJ ΔfucK ΔfucI |
| Chassis 5 | BL21 (λDE3) lacZ (E461A) ΔwacJ ΔfucK ΔfucI |
| Chassis 6 | BL21 (λDE3) lacZ (E537A) ΔwacJ ΔfucK ΔfucI |

### Example 3 Functional Assay of lacZ Gene and Construction of Vector Containing pfkp+pfucT Gene

(1) Determination of lacZ Gene Activity

The determination method of lacZ gene function and enzymatic activity was detected by β-galactosidase reporter gene detection kit (Beyotime), and detailed operation steps are shown in the kit instructions.

(2) Construction of pfkp+pfucT Vector

The fucT gene (GenBank: AF076779) of Helicobacter pylori and the fkp gene (GenBank: AAX45030.1) from Bacteroides fragilis were synthesized by Suzhou Genewiz., China. The two genes were ligated into plasmid pETduet-1 with BamH I—Hind III and Nde I—Xho I as restriction sites, respectively, to obtain the recombinant expression vector pET-Fkp-FucT (Amp^{r}).

### Example 4 Fermentation Experiment

The recombinant plasmid pET-Fkp-FucT described in Example 3 was transfected into the above 6 chassis competent cells, recovered at 37° C. for 1 h, and coated on ampicillin-resistant LB plates with a final concentration of 80 μg/mL, cultured at 37° C. for 10-12 h to obtain fermented recombinant bacteria.

A single colony was picked and inoculated into LB medium with a final concentration of 80 μg/mL ampicillin (tryptone 10 g/L, yeast powder 5 g/L, NaCl 10 g/L), and cultured for 8-10 h and used as seed liquid for shake flask fermentation.

Then, the seed liquid was placed in a 250 ml conical flask containing 100 mL of fermentation medium at a 1% inoculation amount, and ampicillin with a final concentration of 80 μg/mL was added at the same time. The formula of the fermentation medium was: glycerol 20 g/L, peptone 10 g/L, yeast powder 5 g/L, NaCl 10 g/L; prepared with deionized water. Then the flask was cultured at 25° C. and 220 r/min until OD₆₀₀=0.6-0.8, IPTG with a final concentration of 0.1 mM, L-fucose 5 g/L, and lactose 10 g/L were added for a continuous fermentation for 72 h.

After the fermentation, the production of bacterial extracellular 2′-FL and the remaining amount of lactose in the fermentation broth were determined by high performance liquid chromatography (HPLC).

At the same time, the fermentation was completed, and intracellular β-galactosidase activity was measured using the method of Example 3.

Firstly, 2 mL of the fermentation broth was centrifuged at 12,000 rpm for 10 min, the supernatant was collected, passed through a 0.22 μm filter, and the concentrations of extracellular 2′-FL and lactose were detected by HPLC. The results of HPLC detection of 2′-FL and lactose content are shown in the table below.

As shown in the experimental results in Table 5 below, β-galactosidase activity of the active site-specific mutation is much lower than that of lacZ(ΔM15), while the molar yield of 2′-FL is much higher than that operated by lacZ(ΔM15). It is indicated that the gene manipulation mode of active site-specific mutation effectively ensures that the acceptor lactose is not metabolized by the host, thereby increasing the yield of 2′-FL.

TABLE 5
Results of fermentation experiments

| | | Lactose | | | | |
|---|---|---|---|---|---|---|
| | | residue | | | | |
| | Lactose | in | The amount | | molar | |
| | initial | fermen- | of lactose | | yield | |
| | concen- | tation | consumed by | 2′-FL | (2′-FL | β-galacto- |
| | tration | broth | bacteria | yield | mol/lac | sidase |
| | (g/L) | (g/L) | (g/L) | g/L | mol) | activity |
| Chassis 1 | 10 | 9.6 | 0.32 | 0.12 | 0.21 | 300 |
| (control) | | | | | | |
| Chassis 2 | 10 | 9.63 | 0.02 | 0.49 | 0.95 | 8 |
| Chassis 3 | 10 | 9.59 | 0.02 | 0.56 | 0.96 | 6 |
| Chassis 4 | 10 | 6.96 | 2.6 | 0.56 | 0.13 | 350 |
| (control) | | | | | | |
| Chassis 5 | 10 | 8.72 | 0.04 | 1.76 | 0.97 | 6 |
| Chassis 6 | 10 | 8.76 | 0.07 | 1.65 | 0.94 | 7 |

The above-mentioned examples are only preferred examples for fully illustrating the present invention, and the protection scope of the present invention is not limited thereto. Equivalent substitutions or transformations made by those skilled in the art based on the present invention are all within the protection scope of the present invention. The protection scope of the present invention is subject to the claims.

1. A genetically engineered bacteria, characterized in that, it is an E. coli with integrated lysogenic λDE3, and lacZ gene is completely inactivated, but does not affect exogenous protein expression of the genetically engineered bacteria, wherein β-galactosidase encoded by lacZ gene has E461A and/or E537A mutations compared with β-galactosidase encoded by wild-type lacZ gene; wherein wacJ, fucK and fucI genes are further knocked out in the genetically engineered bacteria; and wherein the genetically engineered bacteria further comprise fucT gene and fkp gene.

2. The genetically engineering bacteria according to claim 1, wherein codon for amino acid A is GCG.

3. (canceled)

4. The genetically engineered bacteria according to claim 1, wherein the fucT gene is derived from Helicobacter pylori; and/or the fkp gene is derived from Bacteroides fragilis.

5. The genetically engineered bacteria according to claim 1, wherein the E. coli is E. coli DH5α (λDE3), E. coli BL21 (λDE3), E. coli BL21 Star (DE3), or E. coli JM109 (λDE3).

6. A method for culturing genetically engineered bacteria, comprising culturing the genetically engineered bacteria according to claim 1 in a medium.

7. (canceled)

8. A method for preparing 2′-fucosyllactose, wherein the genetically engineered bacteria according to claim 1 is adopted, L-fucose and lactose are added in fermentation medium for fermentation to obtain the 2′-fucosyllactose.

9. (canceled)

10. Use of the genetically engineered bacteria according to claim 1 in the production of 2′-fucosyllactose.

11. The genetically engineered bacteria according to claim 4, wherein the fucT gene has GenBank accession number AF076779 and/or the fkp gene has GenBank accession number AAX45030.1.

12. The genetically engineered bacteria according to claim 4, wherein the fucT gene and the fkp gene are integrated into separate or same backbone plasmid.

13. The genetically engineered bacteria according to claim 12, wherein the backbone plasmid is pETduet-1.

14. The method according to claim 6, wherein the medium is E. coli conventional medium.

15. The method according to claim 6, wherein the medium is LB, SOB, SOC, 2×YT, TB, or SB medium.

16. The method according to claim 8, wherein the fermentation medium of the genetically engineered bacteria comprises 20 g/L glycerol or glucose, 10 g/L peptone, 5 g/L yeast powder, and 10 g/L NaCl.

17. The method according to claim 8, wherein when the genetically engineered bacteria are cultured to OD₆₀₀=0.5˜1.0, preferably 0.6˜0.8, a final concentration of 0.1-0.3 mM IPTG, such as 0.1 mM IPTG, 5 g/L L-fucose, and 10 g/L lactose are added.
